(19) United States
(12) Patent Application Publication
(10) Pub. No.: US 2023/0263649 A1
(43) Pub. Date: Aug. 24, 2023

(54) ENDOVASCULAR PROSTHESIS DELIVERY SYSTEM

(71) Applicant: EVASC NEUROVASCULAR LIMITED PARTNERSHIP, Vancouver (CA)

(72) Inventors: DONALD R. RICCI, Vancouver (CA); STEPHEN LUONG, Vancouver (CA); KENNETH BARRY, Vancouver (CA); ADAM TYMAN, North Vancouver (CA); CHRISTOPHER G.M. KEN, San Carlos, CA (US)

(21) Appl. No.: 17/802,825

(22) PCT Filed: Mar. 1, 2021
(86) PCT No.: PCT/CA2021/050258

§ 371 Date: Aug. 26, 2022

Related U.S. Application Data
(60) Provisional application No. 63/100,125, filed on Feb. 28, 2020.

(51) Int. Cl. A61F 2/962

(52) U.S. Cl. CPC A61F 2/962; A61F 2002/9505

(57) ABSTRACT

This is disclosed an endovascular prosthesis delivery system. The delivery system comprises an elongate delivery device comprising a delivery device longitudinal axis. The elongate delivery device is coupled to an endovascular prosthesis via a connection portion. The connection portion is configured to be detachable from the endovascular prosthesis or the delivery device upon application an electric current to the delivery device. The endovascular prosthesis in an unsheathed state and the elongate delivery device being rotatable with respect to one another about the delivery device longitudinal axis.

### CROSS-REFERENCE TO RELATED APPLICATION

The present application claims the benefit under 35 U.S.C. §119(e) of provisional patent application S.N. 63/100,125, filed Feb. 28, 2020, the contents of which are hereby incorporated by reference.

### BACKGROUND OF THE INVENTION

### Field of the Invention

In one of its aspects, the present invention relates to an endovascular prosthesis delivery system. In another of its aspects, the present invention relates to a method of treating an aneurysm in a patient. Other aspects of the invention will be apparent to those of skill in the art having in hand the present specification.

### Description of the Prior Art

As is known in the art, an aneurysm is an abnormal bulging outward in the wall of an artery. In some cases, the bulging may be in the form of a smooth bulge outward in all directions from the artery - this is known as a “fusiform aneurysm”. In other cases, the bulging may be in the form of a sac arising from an arterial branching point or from one side of the artery - this is known as a “saccular aneurysm”.

While aneurysms can occur in any artery of the body, it is usually those which occur in the brain which lead to the occurrence of a stroke. Most saccular aneurysms which occur in the brain have a neck which extends from the cerebral blood vessel and broadens into a pouch which projects away from the vessel.

The problems caused by such aneurysms can occur in several different ways. For example, if the aneurysm ruptures, blood enters the brain or the subarachnoid space (i.e., the space closely surrounding the brain) - the latter is known as an aneurysmal subarachnoid hemorrhage. This is followed by one or more of the following symptoms: headache, nausea, vomiting, double vision, neck stiffness and loss of consciousness. Aneurysmal subarachnoid hemorrhage is an emergency medical condition requiring immediate treatment. Indeed, 10-15% of patients with the condition die before reaching the hospital for treatment. More than 50% of patients with the condition will die within the first thirty days after the hemorrhage. Of those patients who survive, approximately half will suffer a permanent stroke. Some of these strokes occur one to two weeks after the hemorrhage itself from vasospasm in cerebral vessels induced by the subarachnoid hemorrhage.

Aneurysms also can cause problems which are not related to bleeding although this is less common. For example, an aneurysm can form a blood clot within itself which can break away from the aneurysm and be carried downstream where it has the potential to obstruct an arterial branch causing a stroke (e.g., an ischemic stroke). Further, the aneurysm can also press against nerves or the adjacent brain (this has the potential of resulting in paralysis or abnormal sensation of one eye or of the face, seizures or other neurologic symptoms).

Given the potentially fatal consequences of the aneurysms, particularly brain aneurysms, the art has addressed treatment of aneurysms using various approaches.

Generally, aneurysms may be treated from outside the blood vessels using surgical techniques or from the inside using endovascular techniques (the latter falls under the broad heading of interventional (i.e., non-surgical) techniques).

Surgical techniques usually involve a craniotomy requiring creation of an opening in the skull of the patient through which the surgeon can insert instruments to operate directly on the brain. In one approach, the brain is retracted to expose the vessels from which the aneurysm arises and then the surgeon places a clip across the neck of the aneurysm thereby preventing arterial blood from entering the aneurysm. If there is a clot in the aneurysm, the clip also prevents the clot from entering the artery and obviates the occurrence of a stroke. Upon correct placement of the clip the aneurysm will be obliterated in a matter of minutes. Surgical techniques historically have been the most common treatment for aneurysms. Unfortunately, surgical techniques for treating these conditions are regarded as major surgery involving high risk to the patient and necessitate that the patient be in a condition to have even a chance to survive the procedure.

As mentioned above, endovascular techniques are non-surgical techniques and are typically performed in an angiography suite using a catheter delivery system. Specifically, known endovascular techniques involve using the catheter delivery system to pack the aneurysm with a material which prevents arterial blood from entering the aneurysm leading to obliteration of the aneurysm - this technique is broadly known as embolization.

One example of such an approach is the Guglielmi Detachable Coil which involves intra-aneurysmal occlusion of the aneurysm via a system which utilizes a platinum coil attached to a stainless steel delivery wire and electrolytic detachment. Thus, once the platinum coil has been placed in the aneurysm, it is detached from the stainless steel delivery wire by electrolytic dissolution. Specifically, the patient’s blood and the saline infusate act as the conductive solutions. The anode is the stainless steel delivery wire and the cathode is the ground needle which is placed in the patient’s groin. Once current is transmitted through the stainless steel delivery wire, electrolytic dissolution will occur in the uninsulated section of the stainless steel detachment zone just proximal to the platinum coil (the platinum coil is of course unaffected by electrolysis).

Other approaches to fill the aneurysm sac involve the use of materials such as cellulose acetate polymer.

While these endovascular approaches have advanced the art, they are disadvantageous. Specifically, the risks of these endovascular approaches include rupturing the aneurysm during the procedure or causing a stroke (e.g., an ischemic stroke) due to distal embolization of the device or clot from the aneurysm. Additionally, concern exists regarding the long term results of endovascular aneurysm obliteration using these techniques. Specifically, there is evidence of intra-aneurysmal rearrangement of the packing material and reappearance of the aneurysm on follow-up angiography.

One particular type of brain aneurysm which has proven to be very difficult to treat, particularly using the surgical clipping or endovascular embolization techniques discussed above occurs at bifurcations, where a parent artery branches into two smaller branch arteries. An example of this type of aneurysm is one that occurs at the terminal bifurcation of the basilar artery. Successful treatment of bifurcation aneurysms (e.g., using a surgical clip) is very difficult due, at least in part, to the imperative requirement that all the brainstem perforating vessels be spared during surgical clip placement.

Unfortunately, there are occasions when the size, shape and/or location of an aneurysm make both surgical clipping and endovascular embolization not possible for a particular patient. Generally, the prognosis for such patients is not good.

Accordingly, while the prior art has made advances in the area of treatment of aneurysms, there is still room for improvement, particularly in endovascular embolization since it is such an attractive alternative to major surgery.

In Intenational Publication Number WO 99/40873 [Marotta et al. (Marotta)], published Aug. 19, 1999, there is taught a novel endovascular approach useful in blocking of an aneurysmal opening, particularly those in saccular aneurysms, leading to obliteration of the aneurysm. The approach is truly endovascular in that, with the endovascular prosthesis taught by Marotta, there is no requirement to pack the aneurysmal sac with a material (e.g., such is used with the Guglielmi Detachable Coil). Rather, the endovascular prosthesis taught by Marotta operates on the basis that it serves to block the opening to the aneurysmal sac thereby obviating the need for packing material. Thus, the endovascular prosthesis taught by Marotta is an important advance in the art since it obviates or mitigates many of the disadvantages of the prior art. The endovascular prosthesis taught by Marotta comprises a leaf portion capable of being urged against the opening of the aneurysm thereby closing the aneurysm. In the endovascular prosthesis taught by Marotta, the leaf portion is attached to, and independently moveable with respect to, a body comprising at least one expandable portion. The expandable portion is expandable from a first, unexpanded state to a second, expanded state with a radially outward force thereon. Thus, the body serves the general purpose of fixing or anchoring the endovascular prosthesis in place at a target body passageway or vascular lumen in the vicinity at which the aneurysmal opening is located and the leaf portion serves the purpose of sealing the aneurysmal opening thereby leading to obliteration of the aneurysm. Thus, as taught by Marotta, the leaf portion functions and moves independently of the body of the endovascular prosthesis.

International Publication Numbers WO 2012/145823A1 and WO 2012/145836 [both in the name of Tippett et al. (Tippett #1)] teach an endovascular prosthesis and an endovascular prosthesis delivery device. The endovascular prosthesis disclosed by Tippett #1 is an improvement over the endovascular device disclosed by Marotta in that the former is designed to allow the physician to be able to retrieve the device so that it may be repositioned for optimum placement prior to detachment from the delivery system. The endovascular prosthesis delivery device disclosed by Tippett can take the form of a number of different embodiments.

International Publication Number WO 2018/058254A1 [Fung et al. (Fung)] teaches an endovascular prosthesis delivery device which is an improvement of the device taught by Tippett #1. The endovascular prosthesis delivery device taught by Fung comprises a combination of a delivery frame element and a hub insert element that are secured to one another by a first retention element. At a distal portion of the delivery frame element, there is a prosthesis attachment zone for coupling to an endovascular prosthesis. When it is desired to detach the endovascular prosthesis, the first retention element is mechanically broken in a manner to allow relative movement between the hub insert element and the delivery frame element. A pull wire assembly is secured with respect to the hub insert element and comprises a pull wire which is coupled to the endovascular prosthesis in the prosthesis attachment zone of the delivery frame element. Once the first retention element is mechanically broken by the physician (this is done when the endovascular prosthesis is in the correct position for detachment), the physician can then retract the hub insert which has the effect of retracting pull wire from the prosthesis attachment zone of the delivery frame element. The endovascular prosthesis and the endovascular prosthesis delivery device are now detached from one another and the latter may be withdrawn from the patient.

While the device taught by Fung is a significant improvement in the art, there is room for improvement.

First, due the number of elements at the distal end (see FIGS. 7-8 of Fung) and the annular design of the illustrated embodiments of the device, it is difficult to produce a low profile delivery device (e.g., less than 0.034 inches). When the profile of the device is 0.034 inches, it is primarily indicated for large vessels only such as second order basilar arteries or first order carotid arteries. This represents only 12-15% of the neurovasculature in which aneurysms may occur.

Second, during clinical development work with the Fung delivery device using the delivery approach taught by International Publication Number WO 2014/066982 [Tippett #2] (see Paragraph [0042] of Fung), it was necessary to use two guidewires as illustrated in FIGS. 11-16 of Tippett #2. The is problematic for two reasons: (i) it adds an extra step for the physician to undertake to deliver the prosthesis, and (ii) the delivery of the second guidewire to second secondary passageway of the bifurcation is challenging due to the small distance (3-4 mm) from the distal end of the hypotube delivery device to the opening of second secondary passageway (see delivery device 200 and secondary passageway 20 in FIG. 14 of Tippett #2].

Third, with reference to FIG. 12 of Fung, there is relatively limited axial rotational movement of elongate endovascular prosthesis 100 about pull wire 45 via attachment loop 95 - i.e., rotation of a longitudinal axis of elongate endovascular prosthesis 100 about the longitudinal axis of pull wire 45 is limited to about 130°. This limits the freedom of positioning the prosthesis by the physician.

While electrolytic detachment of a prosthesis from a delivery system is generally known, to the knowledge of the present inventions, there is no known delivery system that can deliver an endovascular prosthesis (usuing eletrolytic detachment or otherwise) while obviating or mitigating the problems discussed above.

Accordingly, there remains a need in the art for an endovascular prosthesis delivery device that overcomes at least some if not all of the above-mentioned problems with the Fung delivery device. It would be further desirable if such an endovascular prosthesis delivery device was relatively simple to manufacture and use to deliver and implant an endovascular prosthesis. It would be highly advantageous if relatively simple and reliable mechanism was available to detach the endovascular prosthesis from the delivery device.

### SUMMARY OF THE INVENTION

It is an object of the present invention to obviate or mitigate at least one of the above-mentioned disadvantages of the prior art.

It is another object of the present invention to provide a novel endovascular prosthesis delivery system.

Accordingly, in one of its aspects, the present invention relates to an endovascular prosthesis delivery system comprising an elongate delivery device comprising a delivery device longitudinal axis, the elongate delivery device being coupled to an endovascular prosthesis via a connection portion, the connection portion configured to be detachable from the endovascular prosthesis or the delivery device upon application an electric current to the delivery device, the endovascular prosthesis in an unsheathed state and the elongate delivery device being rotatable with respect to one another about the delivery device longitudinal axis.

In another of its aspects, the present invention relates to a method for delivering an endovascular prosthesis to a bifurcated vasculature in a patient, the bifurcated vasculature comprising a primary passageway and at least one secondary passageway to define an intersection at which is located an aneurysm having an aneurysmal opening, the method comprising the steps of:
(i) advancing a guidewire through the primary passageway into the secondary passageway;
(ii) advancing a catheter surrounding the guidewire through the primary passageway into the secondary passageway;
(iii) removing the guidewire from the patient;
(iv) advancing the present endovascular delivery system (in any of its embodiments) to a distal portion of the catheter;
(v) retracting the catheter with respect to the endovascular prosthesis to expose an anchor portion of the endovascular prosthesis;
(vi) implanting the anchor portion of the endovascular prosthesis in the secondary passageway;
(vii) further retracting the catheter with respect to the endovascular prosthesis to expose a blood occlusion portion of the endovascular prosthesis;
(viii) aligning the blood occlusion portion of the endovascular prosthesis with the aneurysmal opening;
(ix) implanting the blood occlusion portion of the endovascular prosthesis so as to occlude the aneurysmal opening;
(x) applying a current to the elongate delivery device;
(xi) detaching the connection portion from the elongate delivery device; and
(xii) retracting the elongate delivery device and the catheter from the patient.

In another of its aspects, the present invention relates to a method for delivering an endovascular prosthesis to a bifurcated vasculature in a patient, the bifurcated vasculature comprising a primary passageway and at least one secondary passageway to define an intersection at which is located an aneurysm having an aneurysmal opening, the method comprising the steps of:
(i) advancing a guidewire through the primary passageway into the secondary passageway;
(ii) advancing a catheter surrounding the guidewire through the primary passageway into the secondary passageway;
(iii) removing the guidewire from the patient;
(iv) abutting a distal end of the present endovascular prosthesis delivery system (in any of its embodiments) containing a packaging sheath to a proximal end of the catheter;
(v) advancing the elongate delivery device and the endovascular prosthesis to a distal portion of the catheter while maintaining the packaging sheath external to the patient;
(vi) retracting the catheter with respect to the endovascular prosthesis to expose an anchor portion of the endovascular prosthesis;
(vii) implanting the anchor portion of the endovascular prosthesis in the secondary passageway;
(viii) further retracting the catheter with respect to the endovascular prosthesis to expose a blood occlusion portion of the endovascular prosthesis;
(ix) aligning the blood occlusion portion of the endovascular prosthesis with the aneurysmal opening;
(x) implanting the blood occlusion portion of the endovascular prosthesis so as to occlude the aneurysmal opening;
(xi) applying a current to the elongate delivery device;
(xii) detaching the connection portion from the elongate delivery device; and
(xiii) retracting the elongate delivery device and the catheter from the patient.

The term “occlude”, as used throughout this specification, is intended to have a broad meaning and includes obstruct, cover, block and/or close. An endovascular prosthesis used with the present endovascular prosthesis delivery system will typically be configured to initially obstruct an aneursymal opening of a target aneursym. This causes an interrption or reduction of blood flow into the aneurysm leading to thrombosis of blood in the aneurysmal sac and ultimately obliteration of the aneurysm.

Thus, the present inventors have developed a novel endovascular prosthesis delivery system. The subject endovascular prosthesis delivery system comprises a combination of elongate delivery device having a delivery device longitudinal axis. The delivery system further comprises a connection portion at a distal end thereof and an endovascular prosthesis coupled to the connection portion. The connection portion is configured to be detachable from the elongage endovascular prosthesis or the delivery device upon application of an electric current to the delivery device. Importantly, in an unsheathed state, the endovascular prosthesis is configured to be rotatable about the delivery device longitudinal axis. A number of advantages accrue from the present endovascular prosthesis delivery system.

First, unlike conventional endovascular prosthesis delivery devices, the present system can be used to push the endovascular prosthesis to a desired location by torqueing and steering the elongate delivery device. This can be done without the need for any guidewire to guide the delivery device/endovascular prosthesis to the correct location in the vasculature. In essence, the elongate delivery device of the present system itself functions in much the same way as a guidewire.

Second, the present endovascular prosthesis delivery system has a very low profile. For example, the profile of a preferred embodiment of the present endovascular prosthesis delivery system is well below 0.034 inches. This allows for access to almost all of the neurovasculature in which aneurysms may occur (at least significantly more than can be accessed using the Fung device referred to above).

Third, the requirement to use two guidewires in delivery of the endovascular prosthesis taught by Tippett #2 referred to above is avoided. Thus, the extra step for the physician to undertake delivery of the prosthesis is avoided and the challenge associated with delivery of a second guidewire to a second secondary passageway of the bifurcation is avoided.

Fourth, the present endovascular prosthesis delivery system is characterized by being able to achieve axial rotation of the endovascular prosthesis (in an unsheathed state) to a degree far more than can be achieved using the endovascular prosthesis delivery device taught by Fung referred to above (~130°). In a preferred embodiment, the endovascular prosthesis longitudinal axis can be rotated axially a full 360° or more (e.g., multiple complete rotations such as 720° and 1080°) about the delivery device longitudinal axis.

Fifth, the combination of the following preferred features facilitates access to a secondary passageway in a bifurcated vasculature: flexible and/or shaped (e.g., to a particular angle to accommodate the angle subtending the primary and secondary passageway) distal portion of the elongate delivery device, a hinged connection between elongate delivery device and prosthesis (e.g., they are in a gimballed relationship) and the ability for the prosthesis to be prolapsed. The dynamic hinged (e.g., gimballed) relationship between the endovascular prosthesis and the elongate delivery device transitions from a relatively obtuse relationship to a relatively perpendicular relationship to a relatively acute relationship. This is a particular advantage of the present endovascular prosthesis delivery system that is achievable with no additional guidewire while still permitting access the to a secondary body passageway in the bifurcated passageway of the end of endovascular prosthesis coupled to the elongate delivery device.

Sixth, the aligning step in Step (xiii) in Paragraph [0029] and Step (ix) in Paragraph [0030] is typically is in the linear plane. Surprisingly, in a preferred embodiment of the present endovascular prosthesis delivery system, a second highly advantageous alignment is in the rotational plane. This preferred embodiment relates to the situation when the present endovascular prosthesis delivery system is used to deliver a device such as the endovascular prosthesis taught by Tippett #1 and Tippett #2 discussed above - i.e., an endovascular prosthesis having a blood occlusion or leaf portion comprising a spine having ribs connect thereto. It has been unexpectedly discovered by the present inventors that when the present endovascular prosthesis delivery system is used to delivery such an endovascular prosthesis, rotational alignment of the latter occurs such that the spine auto-aligns to the outer curvature of the microcatheter with the result that the spine is beneath the neck of the aneurysm. This occurs reliably and is a serendipitous finding. While not wishing to be bound by any particular theory or mode of action, the present inventors believe this may occur due to a combination of the non-tubular nature of the endovascular prosthesis and the asymmetric mass of the spine (e.g., such as is taught by Tippett #1 and Tippett #2) versus the rib along the semi-circumference of the device.

The present endovascular prosthesis delivery system comprises two general embodiments.

In the first general embodiment, the connection portion (or at least a portion thereof) is configured to be detachable from the elongate delivery device upon application an electric current to the elongate delivery device. In this first general embodiment, the connection portion (or at least a portion thereof) of the elongate delivery device is configured to remain coupled to the endovascular prosthesis after detachment of the connection portion (or at least a portion thereof) from the elongate delivery device. A preferred embodiment of this first general embodiment is illustrated in FIGS. 1-9 and discussed below. In many preferred versions of the first general embodiment, the connection portion is entirely severed from the elongate delivery device upon application an electric current to the elongate delivery device -an example of this is shown in FIG. 9.

In the second general embodiment, the connection portion (or at least a portion thereof) is configured to be detachable from the endovascular prosthesis upon application an electric current to the delivery device. In this second general embodiment, the connection portion (or at least a portion thereof) of the elongate delivery device is configured to remain coupled to the elongate delivery device after detachment of the connection portion (or at least a portion thereof) from the endovascular prosthesis. Preferred embodiments of this second general embodiment is illustrated in FIGS. 18-21. In many preferred versions of the second general embodiment, a retention portion (or at least a portion thereof) comprised in the connection portion is corrodible upon application an electric current to the elongate delivery device. In some preferred versions, the corrodible retention portion may be diposed at a the distal end of the connection portion and distal to a connection point between the elongate endovascular prosthesis and the proximal protion of the connection portion (e.g., as shown in FIGS. 18, 20 and 21). In other preferred version, the corrodible retention portion may be diposed between the proximal end and the distal end of the connection portion such as coterminously with a connection point between the elongate endovascular prosthesis and the connection portion (e.g., as shown in FIG. 19).

The “aligning” step above (Step (viii) in Paragraph [0029] and Step (ix) in Paragraph [0030]) may including torqueing the elongate delivery device of delivery system, either independently or in conjunction with the catheter. This could be done, for example, to seek an alternate secondary passageway of the bifurcated vasculature that would receive a distal portion of the blood occlusion portion of the endovascular prosthesis.

One of the aspects of the invention relates to a method for delivering an endovascular prosthesis to a bifurcated vasculature in a patient and includes the steps of: abutting a distal end of the present endovascular prosthesis delivery system containing a packaging sheath to a proximal end of the catheter; and advancing the elongate delivery device and the endovascular prosthesis to a distal portion of the catheter while maintaining the packaging sheath external to the patient. A particular advantage associated with this aspect of the invention is the physician is provided with the option of retracting the combination of the endovascular prosthesis and the elongate delivery device back in the packaging sheath (extemal to the patient). Once this is done, the physician may then manually alter the elongate delivery device (e.g., in a distal portion thereof), preferably prior to fully sheathing, for example to enhance its overall curvature along its longitudinal axis to optimize directional access to an alternative secondary passageway in the bifurcated vasculature.

### BRIEF DESCRIPTION OF THE DRAWINGS

Embodiments of the present invention will be described with reference to the accompanying drawings, wherein like reference numerals denote like parts, and in which:

FIG. 1 illustrates a perspective view of a preferred embodiment of the first general embodiment of the present endovascular prosthesis delivery system;

FIG. 2 illustrates a top view of a distal portion of the elongate delivery device of the endovascular prosthesis delivery system illustrated in FIG. 1;

FIG. 3 illustrates a side elevation of a distal portion of the elongate delivery device of the endovascular prosthesis delivery system illustrated in FIG. 1;

FIG. 4 illustrates a side elevation of a proximal portion of the core wire element used in elongate delivery device illustrated in FIGS. 2-3;

FIG. 5 illustrates an exploded view of the distal portion of the elongate delivery device illustrated in FIG. 1;

FIGS. 6-7 illustrate sectional views of a distal portion of the elongate delivery device illustrated in FIG. 1;

FIG. 8 illustrates an exploded view of connection of the endovascular prosthesis to the distal portion of the elongate delivery device shown in FIG. 1 prior to detachment;

FIG. 9 illustrates an exploded view of connection of the endovascular prosthesis to the distal portion of the elongate delivery device shown in FIG. 1 after detachment;

FIGS. 10-17 illustrate sequentially the use of the endovascular prosthesis delivery system illustrated in FIG. 1 to implant an endovascular prosthesis in a bifurcated vasculature;

FIGS. 18-21 illustrated the distal region of various preferred embodiments of the second general embodiment of the present endovascular prosthesis delivery system.

### DETAILED DESCRIPTION OF THE PREFERRED EMBODIMENTS

In a first aspect, the present invention relates to an endovascular prosthesis delivery system comprising an elongate delivery device comprising a delivery device longitudinal axis, the elongate delivery device being coupled to an endovascular prosthesis via a connection portion, the connection portion configured to be detachable from the endovascular prosthesis or the delivery device upon application an electric current to the delivery device, the endovascular prosthesis in an unsheathed state and the elongate delivery device being rotatable with respect to one another about the delivery device longitudinal axis.

Preferred embodiments of this first aspect of the invention may include any one or a combination of any two or more of any of the following features:
the endovascular prosthesis is configured to be rotatable with respect to the elongate delivery device at least 180° about a longitudinal axis of the elongate delivery device;
the endovascular prosthesis is configured to be rotatable with respect to the elongate delivery device at least 360° about a longitudinal axis of the elongate delivery device;
the endovascular prosthesis is elongate and comprises a prosthesis longitudinal axis;
the endovascular prosthesis, in an unsheathed state, is coupled to the elongate delivery device such that the prosthesis longitudinal axis is rotatable about delivery device longitudinal axis;
the endovascular prosthesis, in an unsheathed state, is coupled to the elongate delivery device such that the prosthesis longitudinal axis is rotatable at least about 180° about delivery device longitudinal axis;
the endovascular prosthesis, in an unsheathed state, is coupled to the elongate delivery device such that the prosthesis longitudinal axis is rotatable at least about 360° about delivery device longitudinal axis;
the connection portion is configured such at a distal portion thereof extends along delivery device longitudinal axis distally with respect to a connection point between the endovascular prosthesis and the elongate delivery device;
the connection portion comprises a retention element configured to couple the endovascular prosthesis to the elongate delivery device during delivery of the endovascular prosthesis;
at least of portion of the retention portion is corrodible upon application of an electric current to the delivery device to allow the endovascular prosthesis to be detachable from the endovascular prosthesis;
the retention portion is disposed distally with respect to a connection point between the endovascular prosthesis and the elongate delivery device;
the retention portion is substantially T-shaped at a distal end thereof;
the retention portion is substantially ball-shaped at a distal end thereof;
the retention portion is substantially winged-shaped at a distal end thereof;
the retention portion is coterminous with a connection point between the endovascular prosthesis and the elongate delivery device;
the retention portion comprises a wire element;
the connection portion of the elongate delivery device comprises a first retention element, a second retention element and a spacer element to maintain the first retention element and the second retention element in a spaced relationship;
one or both of the first retention element and the second retention element is substantially ball shaped;
the endovascular prosthesis comprises an attachment portion coupled to the spacer element of the connection portion of the elongate delivery device;
the first retention element and the second retention element are configured to retain the attachment portion of endovascular prosthesis therebetween;
the connection portion is configured to be detachable from the endovascular prosthesis upon application an electric current to the delivery device;
the connection portion is configured to be detachable from the elongate delivery device upon application an electric current to the elongate delivery device;
the connection portion of the elongate delivery device is configured to remain coupled to the endovascular prosthesis after detachment of the connection portion from the elongate delivery device;
the connection portion comprises a male portion engaged to a female portion disposed on the endovascular prosthesis;
the female portion comprises a loop portion for receiving the male portion;
the connection portion comprises a female portion engaged to a male portion disposed on the endovascular prosthesis;
an intermediate portion of the elongate delivery device proximal of the connection portion comprises a core wire element coupled to the connection portion of the elongate delivery device;
the core wire element is configured to be non-annular (i.e., solid);
the core wire element is configured to be tubular;
the core wire element has an outer diameter in the range of from about 0.0020 inches to about 0.0140 inches;
the core wire element has an outer diameter in the range of from about 0.0025 inches to about 0.0135 inches;
the core wire element has a variable outer diameter;
the core wire element has a variable outer diameter that decreases from a proximal end to a distal end of the elongate delivery device;
the core wire element has a substantially constant outer diameter;
the intermediate portion of the elongate delivery device is configured to have increasing flexibility in a direction toward the connection portion of the elongate delivery device;
intermediate portion of the elongate delivery device comprises a decreasing diameter in a direction toward the connection portion of the elongate delivery device;
the intermediate portion of the elongate delivery device further comprises an outer tubular element surrounding at least a portion of the core wire element;
the outer tubular element is porous;
the outer tubular element is configured to be in the form of a first coiled element;
the outer tubular element is configured to be in the form of a first mesh element;
the outer tubular element is configured to be radiopaque;
the intermediate portion of the elongate delivery device further comprises an inner tubular element interposed between and secured with respect to the outer tubular element and the core the core wire element;
the inner tubular element is porous;
the inner tubular element is configured to be in the form of a second coiled element;
the inner tubular element is configured to be in the form of a second mesh element;
the intermediate portion of the elongate delivery device further comprises an elongate annular sealing portion coupled to the outer tubular element surrounding a portion of the core wire;
the elongate annular sealing portion is configured to expose a portion of the core wire element proximal to the connection portion of the elongate delivery device;
a distal portion of the elongate annular sealing portion has a stepped cross-section taken along the longitudinal axis of the elongate delivery device (this prevents the detachment zone from closing up and preventing electrolytic detachment);
the elongate annular sealing portion is substantially electrically non-conductive;
the elongate annular sealing portion is low friction and/or is lubricious;
at least a distal portion of the intermediate portion is curved with respect to a longitudinal axis of the elongate delivery device in a resting state of the elongate delivery device;
the intermediate portion of the elongate delivery device is surrounded by a jacket element;
the jacket element is a constructed from a polymer;
the jacket element is substantially electrically non-conductive;
the connection portion of the elongate delivery device is configured to remain coupled to the endovascular prosthesis after detachment of the connection portion from the elongate delivery device;
at least a portion of the connection portion is configured to be radiopaque;
the connection portion is configured to be radiopaque;
the endovascular prosthesis is configured to be self-expanding;
the endovascular prosthesis comprises an anchor portion and a blood occlusion portion;
the outside diameter of the delivery system is less than about 0.2 inches;
the outside diameter of the delivery system is less than about 0.034 inches;
the outside diameter of the delivery system is in the range of from about 0.010 inches to about 0.030 inches;
the outside diameter of the delivery system is about 0.014 inches;
the outside diameter of the delivery system is about 0.018 inches;
the outside diameter of the delivery system is about 0.024 inches;
the elongate delivery device comprises a coating;
the elongate delivery device comprises a hydrophilic coating; and/or
the delivery system further comprises a packaging sheath element surrounding at least a distal portion of the elongate delivery device and all of the endovascular prosthesis.

In a second aspect, the present invention relates to a method for delivering an endovascular prosthesis to a bifurcated vasculature in a patient, the bifurcated vasculature comprising a primary passageway and at least one secondary passageway to define an intersection at which is located an aneurysm having an aneurysmal opening, the method comprising the steps of:
(i) advancing a guidewire through the primary passageway into the secondary passageway;
(ii) advancing a catheter surrounding the guidewire through the primary passageway into the secondary passageway;
(iii) removing the guidewire from the patient;
(iv) advancing the present endovascular delivery system (in any of its embodiments) to a distal portion of the catheter;
(v) retracting the catheter with respect to the endovascular prosthesis to expose an anchor portion of the endovascular prosthesis;
(vi) implanting the anchor portion of the endovascular prosthesis in the secondary passageway;
(vii) further retracting the catheter with respect to the endovascular prosthesis to expose a blood occlusion portion of the endovascular prosthesis;
(viii) aligning the blood occlusion portion of the endovascular prosthesis with the aneurysmal opening;
(ix) implanting the blood occlusion portion of the endovascular prosthesis so as to occlude the aneurysmal opening;
(x) applying a current to the elongate delivery device;
(xi) detaching the connection portion from the elongate delivery device; and
(xii) retracting the elongate delivery device the catheter from the patient.

Preferred embodiments of this second aspect of the present invention may include any one or a combination of any two or more of any of the following features:
Step (viii) comprises axially rotating the elongate delivery device to align the endovascular prosthesis with the aneurysmal opening;
Step (x) comprises applying a current of about 1 mA at about 12 volts for a duration of from about 30 seconds to about 120 seconds;
Step (x) comprises applying a current of about 1 mA at about 12 volts for a duration of from about 30 seconds to about 105 seconds;
Step (x) comprises applying a current of about 1 mA at about 12 volts for a duration of from about 30 seconds to about 75 seconds;
Steps (i) and (ii) are conducted sequentially; and/or
Steps (i) and (ii) are conducted substantially concurrently.

In a third aspect, the present invention relates to a method for delivering an endovascular prosthesis to a bifurcated vasculature in a patient, the bifurcated vasculature comprising a primary passageway and at least one secondary passageway to define an intersection at which is located an aneurysm having an aneurysmal opening, the method comprising the steps of:
(i) advancing a guidewire through the primary passageway into the secondary passageway;
(ii) advancing a catheter surrounding the guidewire through the primary passageway into the secondary passageway;
(iii) removing the guidewire from the patient;
(iv) abutting a distal end of the present endovascular prosthesis delivery system (in any of its embodiments) containing a packaging sheath to a proximal end of the catheter;
(v) advancing the elongate delivery device and the endovascular prosthesis to a distal portion of the catheter while maintaining the packaging sheath external to the patient;
(vi) retracting the catheter with respect to the endovascular prosthesis to expose an anchor portion of the endovascular prosthesis;
(vii) implanting the anchor portion of the endovascular prosthesis in the secondary passageway;
(viii) further retracting the catheter with respect to the endovascular prosthesis to expose a blood occlusion portion of the endovascular prosthesis;
(ix) aligning the blood occlusion portion of the endovascular prosthesis with the aneurysmal opening; and
(x) implanting the blood occlusion portion of the endovascular prosthesis so as to occlude the aneurysmal opening;
(xi) applying a current to the elongate delivery device;
(xii) detaching the connection portion from the elongate delivery device; and
(xiii) retracting the elongate delivery device and the catheter from the patient.

Preferred embodiments of this third aspect of the present invention may include any one or a combination of any two or more of any of the following features:
Step (ix) comprises axially rotating the elongate delivery device to align the endovascular prosthesis with the aneurysmal opening;
Step (xi) comprises applying a current of about 1 mA at about 12 volts for a duration of from about 30 seconds to about 120 seconds;
Step (xi) comprises applying a current of about 1 mA at about 12 volts for a duration of from about 30 seconds to about 105 seconds;
Step (xi) comprises applying a current of about 1 mA at about 12 volts for a duration of from about 30 seconds to about 75 seconds;
Steps (i) and (ii) are conducted sequentially; and/or
Steps (i) and (ii) are conducted substantially concurrently.

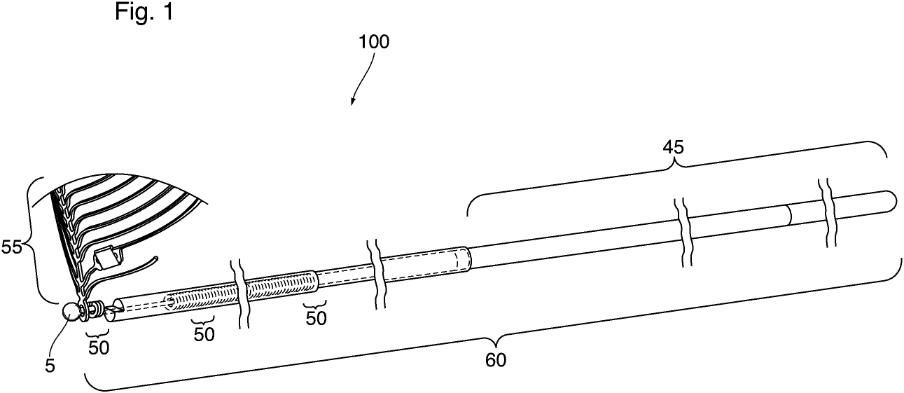

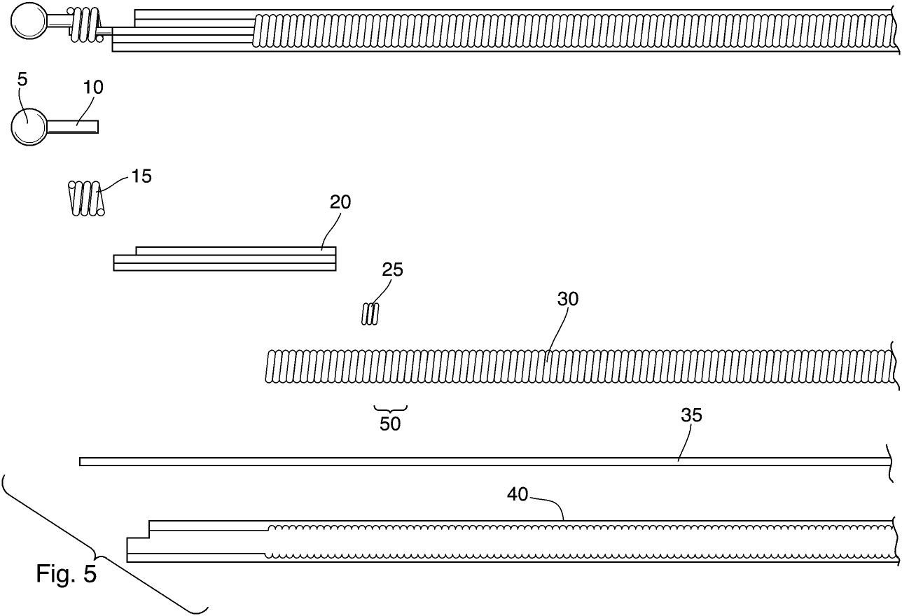

With reference to FIGS. 1-4, there is illustrated a distal portion 100 of a preferred embodiment of the present endovascular prosthesis delivery system.

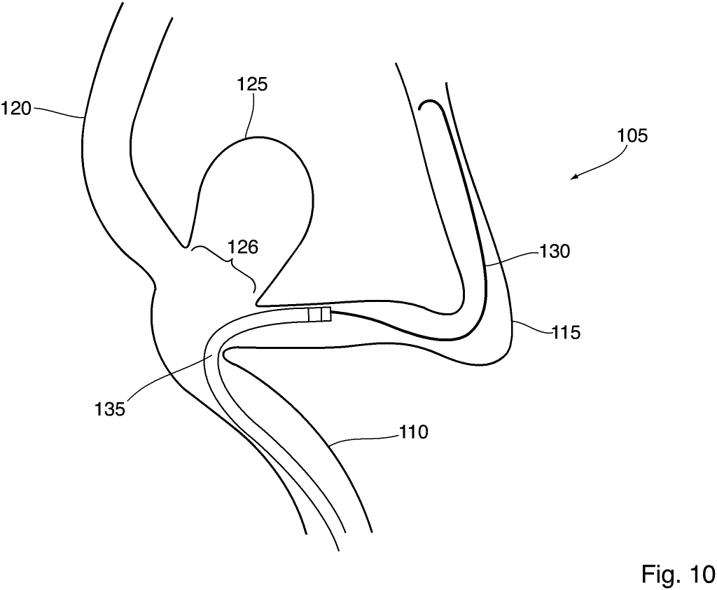

The components in FIGS. 1-4 can be easily understood with reference to FIG. 5 which illustrates the components in an exploded view in relative alignment along a longitudinal axis of proximal portion 100 of the endovascular prosthesis delivery system (some components are listed in FIGS. 10-17):

| Reference Numeral | Component |
|---|---|
| 5 | ball element |
| 10 | ball tip wire element |
| 15 | proximal dumbbell coil element |
| 20 | distal seal element |
| 25 | inner coil element |

| Reference Numeral | Component |
|---|---|
| 30 | outer coil element |
| 35 | core wire element |
| 40 | polymer jacket |
| 45 | PTFE coating |
| 50 | solder points |
| 55 | endovascular prosthesis |
| 56 | anchor portion |
| 57 | blood occlusion portion |
| 60 | elongate delivery device |
| 100 | delivery system |
| 130 | guidewire |
| 135 | microcatheter |

With reference to FIG. 1, there is illustrated an endovascular prosthesis delivery system 100.

Delivery system 100 comprises an elongate delivery device 60 and an endovascular prosthesis 55. Preferably, the endovascular generally comprises an anchor portion and a blood occlusion portion connected to one another. More preferably, the endovascular prosthesis is the one disclosed in any one of Tippett #1 or Tippet #2.

In a preferred embodiment of all aspects of the invention, delivery system 100 further comprises a packaging sheath which is not shown for clarity. The packaging sheath is configured to surround at least a distal portion (e.g., the distal 20 cm to 50 cm distal portion) of the elongate delivery device and all of the endovascular prosthesis. The packaging sheath is conventional.

Elongate delivery device 60 comprises a ball element 5 connected to a ball tip wire element 10 which is preferably connected to a proximal dumbbell coil element 15 element. In a preferred embodiment ball element 5 and ball tip wire element 10 may be a unitary part. Such a unitary part could produced by forming ball element 5 on the end of ball tip wire element 10 by melting/zapping the latter. Alternatively, these elements be produced independently and coupled in a conventional manner. Preferably, one or more of ball element 5, ball tip wire element 10 and proximal dumbbell coil element 15 is made from a radiopaque material (e.g., a platinum-tungsten amalgam).

In the illustrated embodiment, the combination of ball element 5, ball tip wire element 10 and proximal dumbbell coil element 15 define a connection portion for connecting endovascular prosthesis 55 to elongate delivery device 60. Preferably, for all embodiments of the invention, proximal dumbbell coil element 15 joins ball tip wire element 10 to core wire element 35 via soldering, preferably gold-tin solder to create a radiopaque marker.

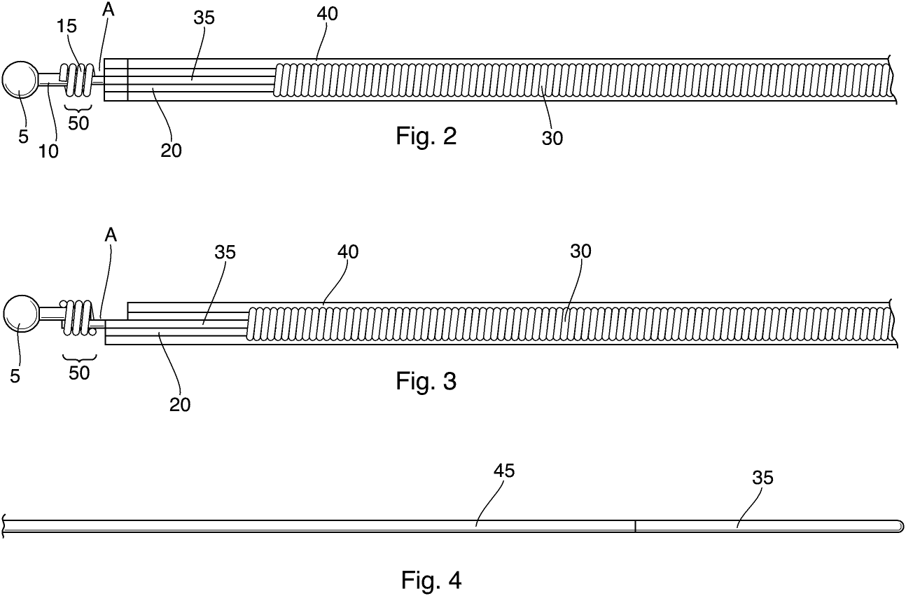

With further reference to FIGS. 2-5, elongate delivery device 60 comprises a distal seal element 20 that is preferably made from an electrically insulating material, more preferably an insulating material that has low friction and is lubricious (e.g., polytetrafluoroethylene or PTFE).

The proximal portion of distal seal element 20 is disposed within an outer coil element 30. Outer coil element 30 is preferably made of a radiopaque material (e.g., a platinum-tungsten amalgam). In the illustrated embodiment, outer coil element 30 is nominally porous. It will be apparent to those of skill in the art that other porous constructions may be used (e.g., mesh).

Disposed within outer coil element 30 is a core wire element 35. Preferably, outer coil element 30 serves to prevent kinking of core wire element 35 and/or improve transfer of torque to core wire element 35 when elongate delivery device 60 is axially rotated. For all embodiments of the invention outer coil element 30 may be substituted with another tubular element (porous or non-porous) that can confer this functionality with respect to core wire element 35 - e.g., a hypotube.

Preferably, for all embodiments of the present invention, core wire element 35 is made of 304V stainless steel and is more preferably covered by a PTFE coating for insulation and lubricity. Preferably, for all embodiments of the present invention, the very proximal end of the core wire element (~8 cm) is bare and more preferably, the distal portion (~45 cm) is tapered for increased flexibility (i.e., the outer diameter of this distal portion of the core wire element decreases in a direction toward the distal end of the core wire element).

Preferably, for all embodiments of the invention, outer coil element 30 is in the form of a platinum coil (~10 cm) which soldered to the distal end of the taper for kink resistance and visibility. Preferably, for all embodiments of the invention, the tapered distal portion (~45 cm) of core wire element 35 and outer coil element 30 are covered with a polymer jacket for insulation and hydrophilic coating for lubricity.

Interposed between outer coil element 30 and core wire element 35 is an inner coil element 25. The positioning of inner coil element 25 serves as one of a number of solder points 50 in elongate delivery device 60. Preferably, the solder (not shown for clarity) is made from a radiopaque material such as gold, gold-tin amalgam and the like.

In the case of inner coil element 25 once solder is applied, outer coil element 30 is secured with respect to core wire element 35. Disposed over outer coil element and distal seal element 20 is a polymer jacket 40 on preferably having a hydrophilic coating applied thereon (not shown for clarity).

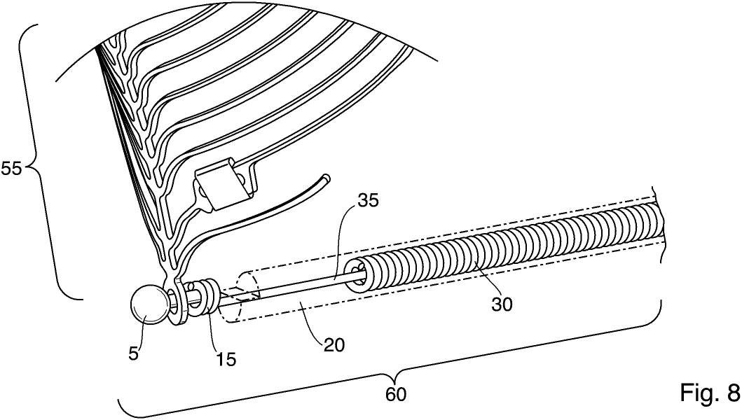

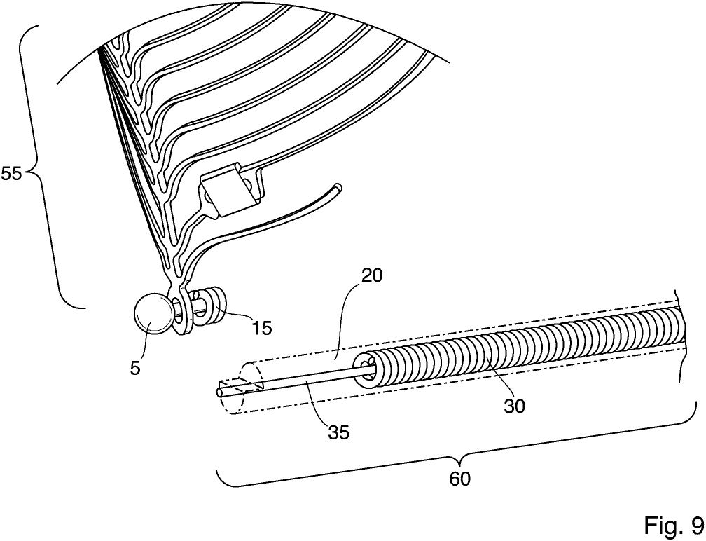

FIG. 8 illustrates endovascular prosthesis 55 coupled to elongate delivery device 60. FIG. 9 illustrates endovascular prosthesis 55 detached from elongate delivery device 60. Detachment is achieved by applying a current to core wire element 35.

As will be understood by those of skill in the art, a short bare portion of the core wire element 35 extends past the distal end distal seal element 20 forming a detachment zone A proximal to proximal dumbbell coil element 15 - see FIGS. 2-3.

As illustrated in FIG. 8, endovascular prosthesis 55 is coupled to elongate delivery device 60 at ball tip wire element 10 and maintained in that position by ball element 5 and proximal dumbbell coil element 15.

As illustrated in FIG. 9, when a suitable current is applied to core wire element 35, to portion of core wire element 35 in detachment zone A corrodes, separating the combination of ball element 5, ball tip wire element 10 and proximal dumbbell coil element 15 coupled to endovascular prosthesis 55 from the rest of elongate delivery device 60.

To achieve detachment as shown in FIG. 9, a circuit is formed when the positive terminal of a DC power source is connected to the proximal end of core wire element 35 and the negative terminal is connected to a needle inserted into the patient’s groin or shoulder. Preferably, for all embodiments of the invention, detachment occurs when DC voltage (~12-15 V) is applied to the proximal end of the core wire element 35 causing a small current (~1 mA) to flow.

The DC power drives the corrosion of the detachment zone which breaks it down into metal ions, resulting in detachment of the combination of ball element 5, ball tip wire element 10 and proximal dumbbell coil element 15 coupled to endovascular prosthesis 55 from the rest of elongate delivery device 60. Preferably, for all embodiments of the invention, by using: (i) a combination of ball element 5, ball tip wire element 10 and proximal dumbbell coil element 15 secured to one another using gold-tin solder, and (ii) platinum to manufacture ball element 5, ball tip wire element 10 and proximal dumbbell coil element 15, corrosion of these elements is obviated mitigated. The insulation over the core wire element 35 and outer coil element 30 isolates the corrosion to the exposed detachment zone and reduces the detachment time. The relatively small size of the detachment zone also minimizes the detachment time.

Further general details on electrolytic detachment can be found in U.S. Pat. 5,122,136 [Guglielmi et al.].

With reference to FIGS. 10-17, the sequence of steps to use delivery system 100 to implant endovascular prosthesis 55 will now be described.

Thus, with reference to FIG. 10, there is illustrated a bifurcated vasculature 105 which comprises a primary passageway 110, a secondary passageway 115 and a secondary passageway 120. At the intersection of primary passageway 110, secondary passageway 115 and secondary passageway 120, there is disposed an aneurysm having an aneurysm opening 126.

As illustrated, a guidewire 130 and a microcatheter 135 are advanced through primary passageway 110 and into secondary passageway 115. Guideware 130 and microcatheter 135 are conventional and the use thereof to advance into secondary passageway 115 is within the purview of a person of ordinary skill in the art.

Once the combination of guidewire 130 and microcatheter 135 are positioned as shown in FIG. 10, guidewire 130 is withdrawn from the patient.

Once guidewire 130 is withdrawn from the patient, the distal end of delivery system 100 described above is abutted to the proximal end of microcatheter 130 (not shown). This can be done, for example, using a rotating haemostasis valve attached to a hub of microcatheter 135. Endovascular prosthesis delivery system 100 is abutted to this portion of the proximal end of microcatheter 135 with the result that the sheathing of the combination of endovascular prosthesis 55 and elongate delivery device 60 essentially is transferred from the packaging sheath to microcatheter 135.

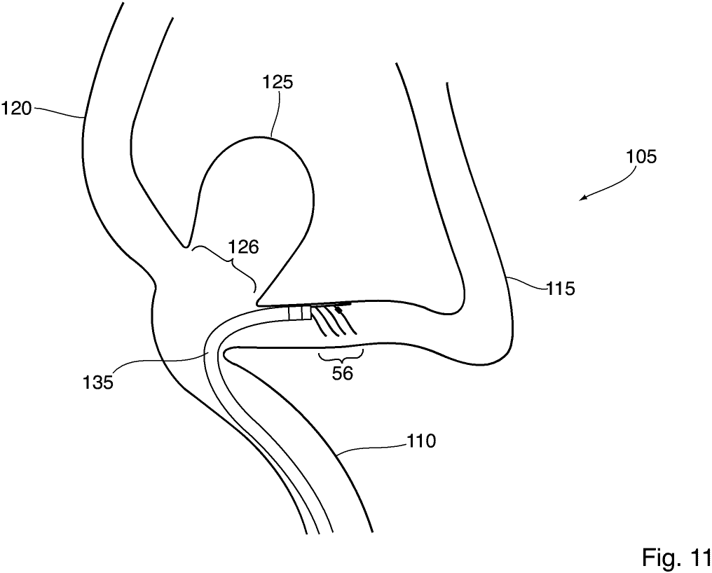

With reference to FIG. 11, the combination of endovascular prosthesis 55 and elongate delivery device 60 is advanced to the distal end of microcatheter 135. Thereafter, microcatheter 135 can be retracted such that an anchor portion 56 of endovascular prosthesis 55 is exposed at the distal end of microcatheter 135.

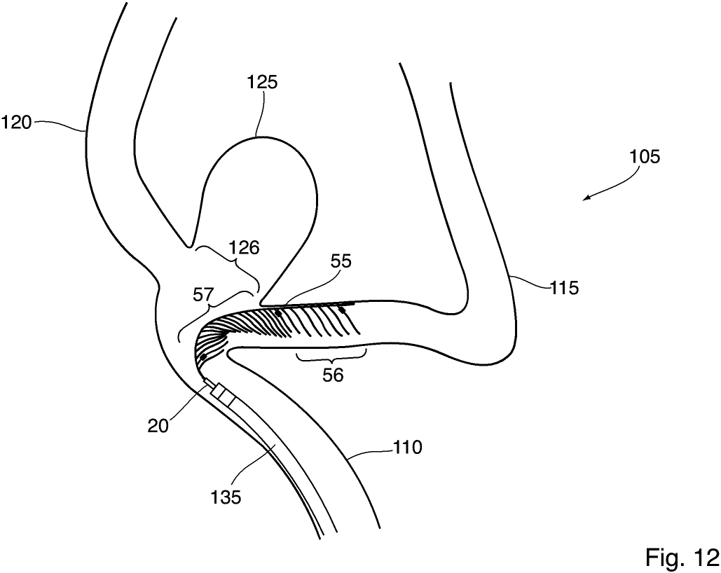

With reference to FIG. 12, once anchor portion 56 is in position, microcatheter 135 is continuously retracted to expose a blood occlusion element 57 of endovascular prosthesis 55. As shown in FIG. 12, a small portion of distal seal element 20 emanates from the distal end of microcatheter 135.

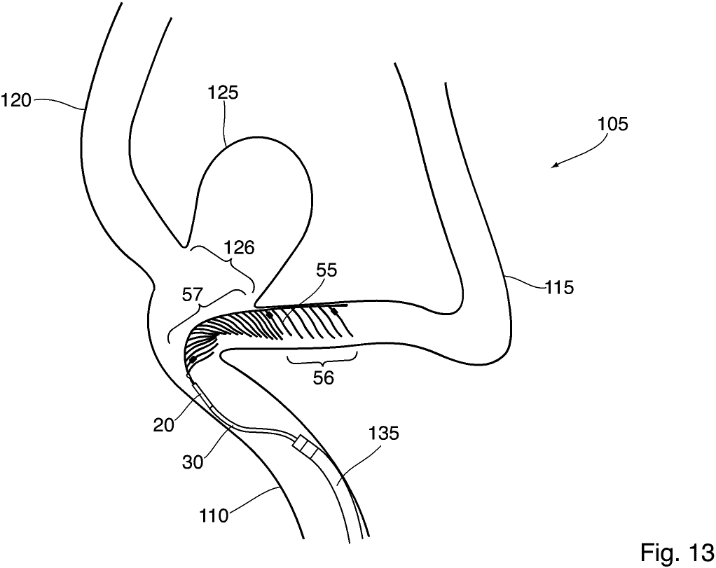

With reference to FIG. 13, microcatheter 135 is further retracted to expose additional length of elongate device 60, namely all of distal seal element 20 and a large portion of outer coil element 30 (it will be appreciated by those of skill in the art that polymer jacket 40 is not shown in FIGS. 12 and 13 for clarity purposes).

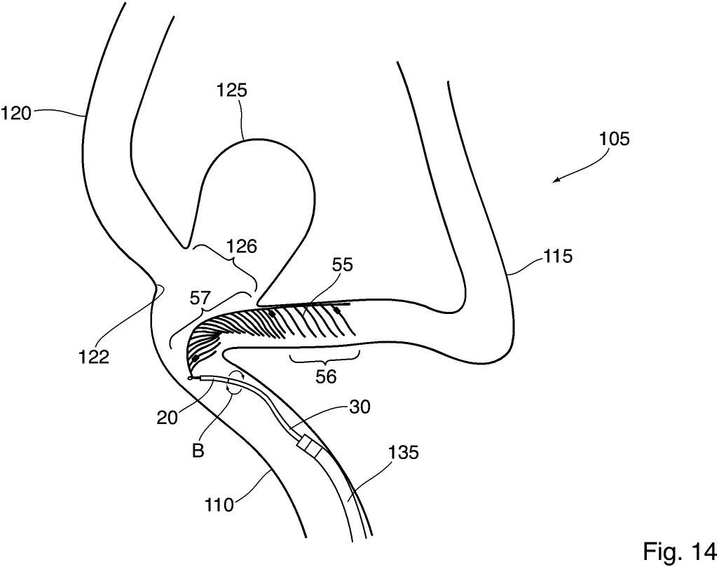

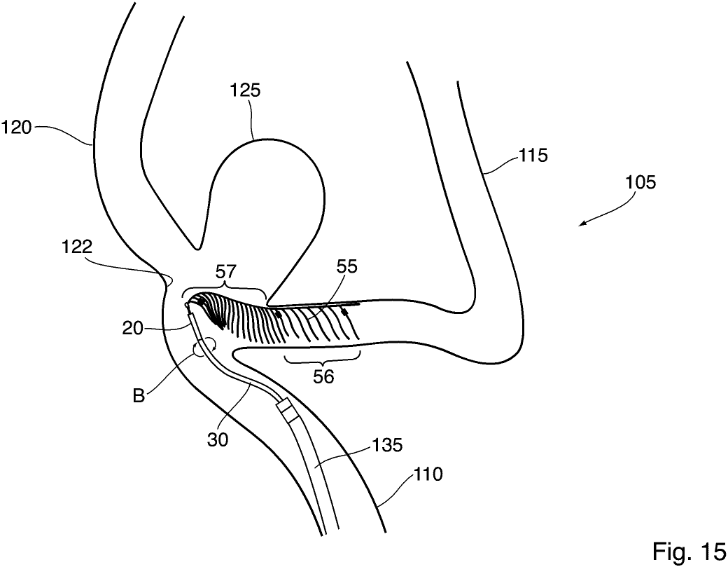

By retracting microcatheter 135 to expose the additional length of elongate delivery device 60 as shown in FIG. 13, the physician is then able to torque elongate delivery device 60 axially as shown by arrow B in FIGS. 14 and 15. This creates the illustrated effect of shifting the connection between endovascular prosthesis 55 and elongate delivery device in an upward direction so as to clear a vasculature shoulder 122 (or a small branch or a perforator vessel or a portion of the lumen) between primary passageway 110 and secondary passageway 120.

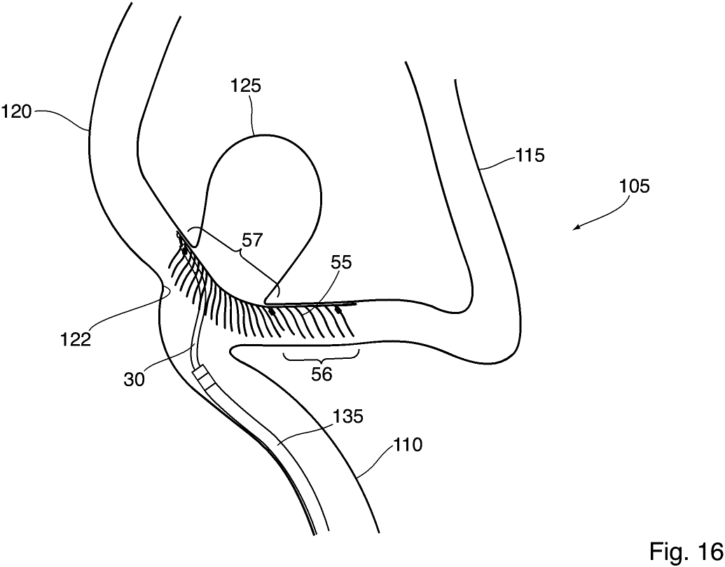

This achieves proper alignment of a blood occlusion portion 57 of endovascular prosthesis 55 with respect to aneurysmal opening 126. Once this alignment is achieved, the distal end of elongate delivery device 60 is advanced to place blood occlusion portion 57 of endovascular portion 55 across aneurysmal opening 126 such that a distal portion of blood occlusion portion 57 is advanced into secondary passageway 120 - see FIG. 16. In all embodiments of the invention, the endovascular prosthesis plays a role in gaining second branch access (e.g., by prolapsing) as described in Paragraph [0037] above.

FIGS. 13-16 illustrate the dynamic hinged (e.g., gimballed) relationship between endovascular prosthesis 55 and elongate delivery device 60 from a relatively obtuse relationship (FIG. 13) to a relatively perpendicular relationship (FIG. 14) to a relatively acute relationship (FIGS. 15-16). This is a particular advantage of all embodiments of the present endovascular prosthesis delivery system that is achievable with no additional guideware while still permitting access the to secondary body passageway 120 of the end of endovascular prosthesis coupled to elongate delivery device 60.

At any time up to this point, the physician may retract endovascular prosthesis 55 into microcatheter 135 to reposition the former in bifurcated vasculature as described in more detail in Tippett #1 and Tippett #2.

Further, at any time up to this point, the physician may retract the combination of the endovascular prosthesis 55 and the elongate delivery device 60 back into the packaging sheath (external to the patient). Once this is done, the physician may then manually alter endovascular prosthesis 55, for example, to enhance its overall curvature along its longitudinal axis to optimize directional access to secondary passageway 120 in bifurcated vasculature 105. Thereafter, the sequence of steps illustrated and described above with respect to FIGS. 11-16 may be repeated.

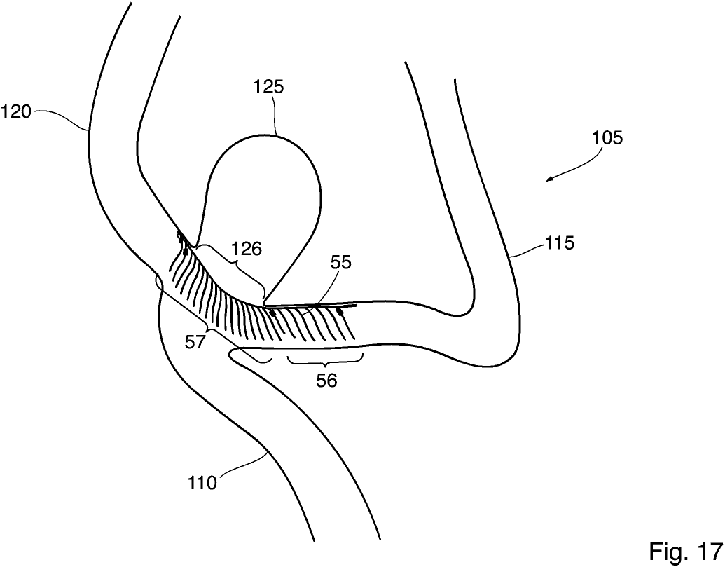

Next, the electrolytic detachment as described above is commenced resulting in detachment of endovascular prosthesis 55 (with the combination of ball element 5, ball tip wire element 10 and proximal dumbbell coil element 15 still attached to endovascular prosthesis 55). Once detachment is achieved, elongate delivery device 60 is retracted with microcatheter 135 leaving endovascular prosthesis 55 implanted and occluding aneurysmal opening 126 of aneurysm 125 - see FIG. 17.

Having now described the use of delivery system 100 to implant endovascular prosthesis 55, those of skill in the art will readily appreciate a key advantage of the present invention is the ability to deliver the endovascular prosthesis to the correct location without the need for a guidewire. The guidewire described above is only used to help position microcatheter 135 correctly. Once this is achieved, guidewire 130 is removed and no further guidewire is necessary to deliver endovascular prosthesis 55. This allows for construction of a relatively low profile delivery system which allows for access to significantly more vasculature than can be accessed using the device taught by Fung referred to above. Thus, elongate delivery device 60 functions in many respects as a guidewire.

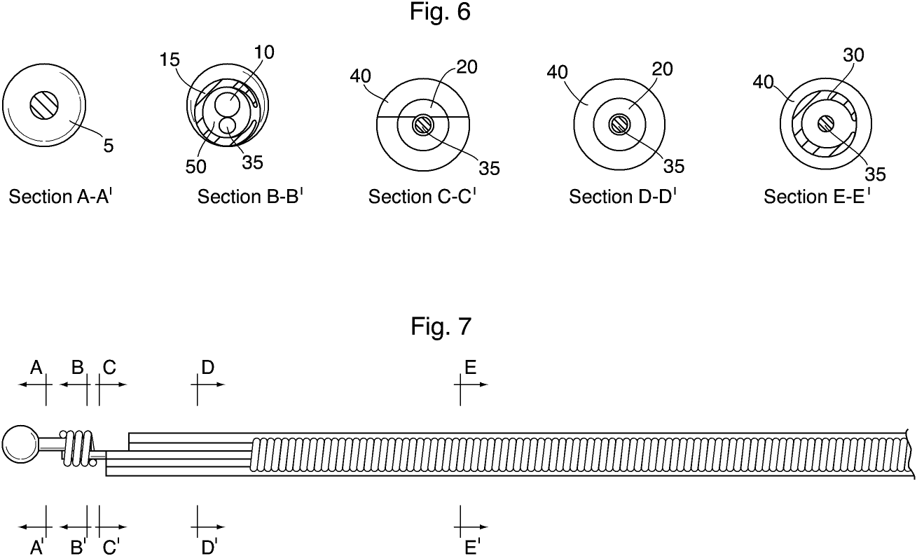

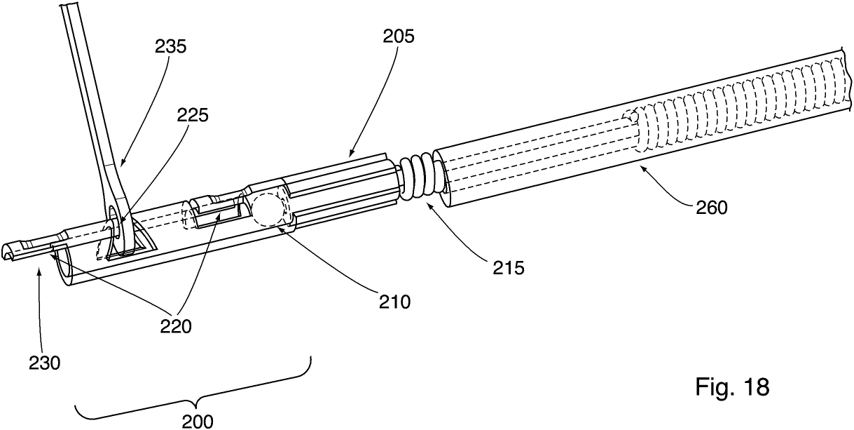

With reference to FIGS. 18-21, there are illustrated various preferred embodiments of the distal region of the second general embodiment of the present endovascular prosthesis delivery system. In each case, the proximal portion of the the second general embodiment of the present endovascular prosthesis delivery system can be constructed using the details discussed above with reference to FIGS. 1-7 (re. the first general embodiment of the present endovascular prosthesis delivery system.

In FIG. 18, a connection portion 200 is disposed at the distal end of the endovascular prosthesis delivery system. Connection portion 200 is coupled to a distal portion of an elongate delivery device 260 via a housing 205 which may be welded or crimped for conduction and for securing a platinum ball tipped wire element 210. A coil element 215 is solder to elongate delivery device 260.

Connection portion 200 comprises a pair of tabs which are bent inward to secure to secure a corrodible detachment wire 225. The distal end 230 of connection portion 200 is bent, round, affixed with a ball tip (not shown for clarity) or otherwise modify to secure detachment wire 225 with respect to the rest of connection portion 200.

An attachment loop 235 which is comprised in the elongate endovascular prosthesis (not shown for clarity) receives detachment wire 225 which secures the elongate endovascular prosthesis to elongate delivery device 260.

The endovascular prosthesis delivery system shown in FIG. 18 is used to deliver the elongate endovascular prothesis using the same general approach described above with reference to FIGS. 10-17. In this case, once the electric current is applied to elongate delivery device 260, the portion of detachment wire 260 connected to attachment loop 235 corrodes allowing the elongate endovascular prosthesis to be detached from detachment wire 260.

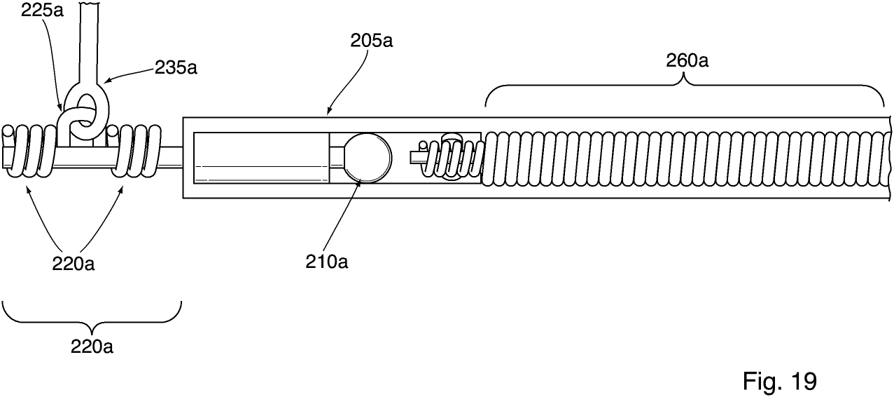

FIG. 19 illustrates a modified connection portion 200a compared to the approach used in FIG. 18 - in FIG. 19 like elements are denoted with the suffix “a”. In FIG. 19, a pair of solder coil connections 220a secure a hand portion 225a with respect to delivery wire 260a.

The endovascular prosthesis delivery system shown in FIG. 19 is used to deliver the elongate endovascular prothesis using the same general approach described above with reference to FIGS. 10-17. In this case, once the electric current is applied to elongate delivery device 260a, handle portion 225a corrodes allowing the elongate endovascular prosthesis to be detached from detachment wire 260a.

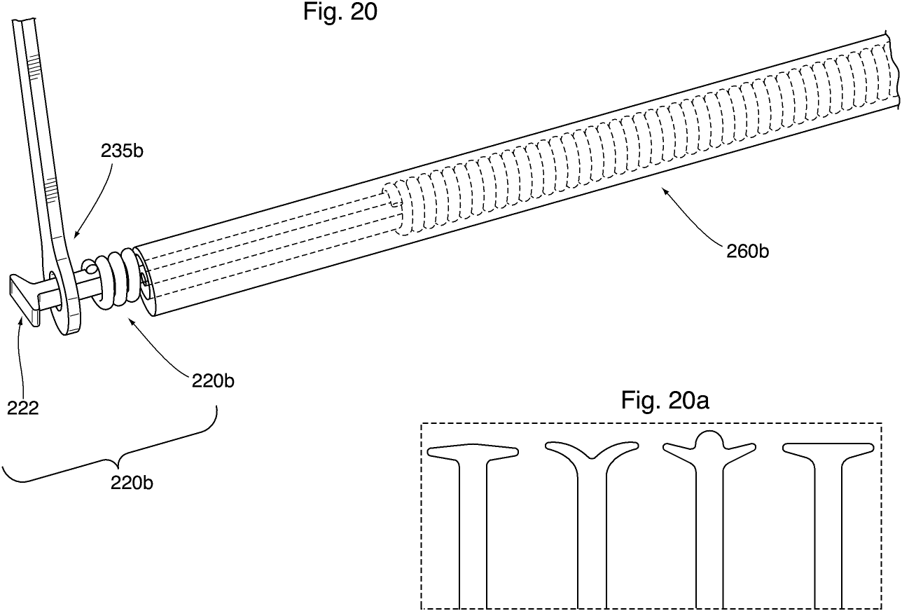

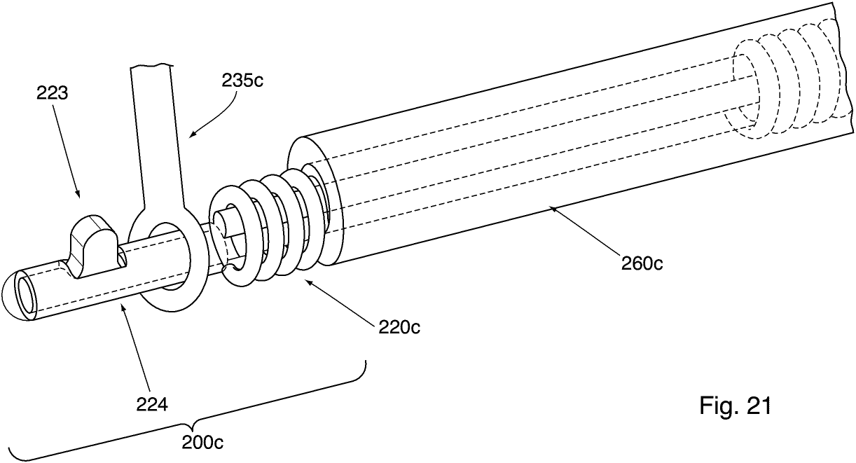

FIG. 20 illustrates a modified connection portion 200b compared to the approach used in FIG. 18 - in FIG. 20 like elements are denoted with the suffix “b”. In FIG. 20, a single solder coil connections 220b is secured with respect to elongate delivery wire 260b. At the distal tip of elongate delivery wire 260b is a T-shaped element 222. T-shaped element 222 is insulated except for the tip projections thereof. Variations to T-shaped element 222 are illustrated in FIG. 20a.

The endovascular prosthesis delivery system shown in FIG. 20 is used to deliver the elongate endovascular prothesis using the same general approach described above with reference to FIGS. 10-17. In this case, once the electric current is applied to elongate delivery device 260b, the tip projections of T-shaped element 222 corrode allowing the elongate endovascular prosthesis to be detached from detachment wire 260b.

FIG. 21 illustrates a modified connection portion 200c compared to the approach used in FIG. 18 - in FIG. 21 like elements are denoted with the suffix “c”. In FIG. 21, a single solder coil connections 220c is secured with respect to elongate delivery wire 260c. At the distal tip of elongate delivery wire 260c is a retention element 223. The distal tip of elongate delivery wire 260c, except for retention element 223, is insulated by a suitable insulating sleeve.

The endovascular prosthesis delivery system shown in FIG. 21 is used to deliver the elongate endovascular prothesis using the same general approach described above with reference to FIGS. 10-17. In this case, once the electric current is applied to elongate delivery device 260c, retention element 223 corrodes allowing the elongate endovascular prosthesis to be detached from detachment wire 260c.

FIGS. 18-21 share the common feature that, after electric current is applied to elongate delivery device, a portion of the connection portion (200, etc.) corrodes allowing the elongate endovascular prosthesis to be detached from detachment wire. The remainder of the connection portion remains coupled to the elongate delivery device.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. An endovascular prosthesis delivery system comprising an elongate delivery device comprising a delivery device longitudinal axis, the elongate delivery device being coupled to an endovascular prosthesis via a connection portion, the connection portion configured to be detachable from the endovascular prosthesis or the delivery device upon application an electric current to the delivery device, the endovascular prosthesis in an unsheathed state and the elongate delivery device being rotatable with respect to one another about the delivery device longitudinal axis.

2. The endovascular prosthesis delivery system defined in claim 1, wherein the endovascular prosthesis is configured to be rotatable with respect to the elongate delivery device at least 180° about a longitudinal axis of the elongate delivery device.

3. (canceled)

4. The endovascular prosthesis delivery system defined in claim 1, wherein the endovascular prosthesis is elongate and comprises a prosthesis longitudinal axis and wherein the endovascular prosthesis, in an unsheathed state, is coupled to the elongate delivery device such that the prosthesis longitudinal axis is rotatable about delivery device longitudinal axis.

5. (canceled)

6. The endovascular prosthesis delivery system defined in claim 4, wherein the endovascular prosthesis, in an unsheathed state, is coupled to the elongate delivery device such that the prosthesis longitudinal axis is rotatable at least about 180° about delivery device longitudinal axis.

7. (canceled)

8. The endovascular prosthesis delivery system defined in claim 1, wherein the connection portion is configured such at a distal portion thereof extends along delivery device longitudinal axis distally with respect to a connection point between the endovascular prosthesis and the elongate delivery device.

9-16. (canceled)

17. The endovascular prosthesis delivery system defined in claim 1, wherein the connection portion of the elongate delivery device comprises a first retention element, a second retention element and a spacer element to maintain the first retention element and the second retention element in a spaced relationship.

18-20. (canceled)

21. The endovascular prosthesis delivery system defined in claim 1, wherein the connection portion is configured to be detachable from the endovascular prosthesis upon application an electric current to the delivery device.

22. The endovascular prosthesis delivery system defined in claim 1, wherein the connection portion is configured to be detachable from the elongate delivery device upon application an electric current to the elongate delivery device.

23. The endovascular prosthesis delivery system defined in claim 1, wherein the connection portion of the elongate delivery device is configured to remain coupled to the endovascular prosthesis after detachment of the connection portion from the elongate delivery device.

24-26. (canceled)

27. The endovascular prosthesis delivery system defined in claim 1, wherein an intermediate portion of the elongate delivery device proximal of the connection portion comprises a core wire element coupled to the connection portion of the elongate delivery device.

28. The delivery system defined in claim 27, wherein the core wire element is configured to be non-annular (i.e., solid).

29-40. (canceled)

41. The endovascular prosthesis delivery system defined in claim 27, wherein at least a distal portion of the intermediate portion is curved with respect to a longitudinal axis of the elongate delivery device in a resting state of the elongate delivery device.

42-47. (canceled)

48. The endovascular prosthesis delivery system defined in claim 1, wherein the endovascular prosthesis comprises an anchor portion and a blood occlusion portion.

49. The endovascular prosthesis delivery system defined in claim 1, wherein the outside diameter of the delivery system is less than about 0.034 inches.

50-56. (canceled)

57. A method for delivering an endovascular prosthesis to a bifurcated vasculature in a patient, the bifurcated vasculature comprising a primary passageway and at least one secondary passageway to define an intersection at which is located an aneurysm having an aneurysmal opening, the method comprising the steps of:
(a) advancing a guidewire through the primary passageway into the secondary passageway;
(b) advancing a catheter surrounding the guidewire through the primary passageway into the secondary passageway;
(c) removing the guidewire from the patient;
(d) advancing the delivery system defined in claim 1 to a distal portion of the catheter;
(e) retracting the catheter with respect to the endovascular prosthesis to expose an anchor portion of the endovascular prosthesis;
(f) implanting the anchor portion of the endovascular prosthesis in the secondary passageway;
(g) further retracting the catheter with respect to the endovascular prosthesis to expose a blood occlusion portion of the endovascular prosthesis;
(h) aligning the blood occlusion portion of the endovascular prosthesis with the aneurysmal opening;
(i) implanting the blood occlusion portion of the endovascular prosthesis so as to occlude the aneurysmal opening;
(j) applying a current to the elongate delivery device;
(k) detaching the connection portion from the endovascular prosthesis or the elongate delivery device; and
(l) retracting the elongate delivery device and the catheter from the patient.

58. The method defined in claim 57, wherein Step (viii) comprises axially rotating the elongate delivery device to align the endovascular prosthesis with the aneurysmal opening.

59. The method defined in claim 57, wherein Step (x) comprises applying a current of about 1 mA at about 12 volts for a duration of from about 30 seconds to about 120 seconds.

60-61. (canceled)

62. A method for delivering an endovascular prosthesis to a bifurcated vasculature in a patient, the bifurcated vasculature comprising a primary passageway and at least one secondary passageway to define an intersection at which is located an aneurysm having an aneurysmal opening, the method comprising the steps of:
(a) advancing a guidewire through the primary passageway into the secondary passageway;
(b) advancing a catheter surrounding the guidewire through the primary passageway into the secondary passageway;
(c) removing the guidewire from the patient;
(d) abutting a distal end of the delivery system defined in claim 1 to a proximal end of the catheter;
(e) advancing the elongate delivery device and the endovascular prosthesis to a distal portion of the catheter while maintaining the packaging sheath external to the patient;
(f) retracting the catheter with respect to the endovascular prosthesis to expose an anchor portion of the endovascular prosthesis;
(g) implanting the anchor portion of the endovascular prosthesis in the secondary passageway;
(h) further retracting the catheter with respect to the endovascular prosthesis to expose a blood occlusion portion of the endovascular prosthesis;
(i) aligning the blood occlusion portion of the endovascular prosthesis with the aneurysmal opening; and
(j) implanting the blood occlusion portion of the endovascular prosthesis so as to occlude the aneurysmal opening;
(k) applying a current to the elongate delivery device;
(l) detaching the connection portion from the endovascular prosthesis or the elongate delivery device; and
(m) retracting elongate delivery device and the catheter from the patient.

63. The method defined in claim 62, wherein Step (ix) comprises axially rotating the elongate delivery device to align the endovascular prosthesis with the aneurysmal opening.

64. The method defined in claim 62, wherein Step (x) comprises applying a current of about 1 mA at about 12 volts for a duration of from about 30 seconds to about 120 seconds.

65-68. (canceled)
